(19) United States
(12) Patent Application Publication
(10) Pub. No.: US 2026/0364262 A1
(43) Pub. Date: Nov. 19, 2026

(54) SYRINGE PLUNGER ROD, SYRINGE BARREL, AND SYRINGE

(71) Applicant: KRUI MEDICAL AB, HELSINGBORG (SE)

(72) Inventor: Per KNUTSSON, Helsingborg (SE)

(73) Assignee: KRUI MEDICAL AB, HELSINGBORG (SE)

(21) Appl. No.: 19/133,878

(22) PCT Filed: Nov. 10, 2023
(86) PCT No.: PCT/SE2023/051139

§ 371 Date: May 29, 2025

(30) Foreign Application Priority Data
Nov. 29, 2022 (SE) 2251387-3

(51) Int. Cl. A61M 5/315; A61M 5/31

(52) U.S. Cl. CPC A61M 5/31513; A61M 5/3134; A61M 2005/3131

(57) ABSTRACT

A syringe plunger rod (100) extending along a longitudinal central axis (A) is provided. The syringe plunger rod (100) comprises a distal sealing plug (102) for sealing engagement with a syringe barrel (200), and a proximal elongated shaft (103) 5 comprising a proximal end (104) adapted to receive an applied force along the longitudinal axis (A). The sealing plug (102) is made of an elastic material and a sealing plug distal surface (105) comprises concentric steps (106), wherein the concentric steps (106) are climbing laterally. A syringe barrel (200) is also provided. The syringe barrel (200) comprises a needle hub (204) provided with longitudinal needle press-fit channel 10 (206). The press-fit channel (206) is flush with the distal plunger socket wall (202), such that the press-fit channel (206) extends proximally into the volume (V). A hollow needle (205) is press fitted in the press-fit channel (206).

### TECHNICAL FIELD

The present invention relates to a syringe plunger rod, a syringe barrel, and a syringe comprising a syringe plunger rod and a syringe barrel.

### BACKGROUND

Injections are among the most common health care procedures, with as much as 22 billion injections administered globally each year. During 2021, the Covid-19 mass vaccination will require even further vast amounts of syringes to conduct the injections.

Syringes known in the art may be pre-filled syringes, delivered already holding a medicament/vaccine, or conventional syringes which are empty and filled on site with the drug to be administered to the patient. Pre-filled syringes are made from either glass or polymeric materials. However, they are not manufacturing process is not cost effective. Further, the glass syringes are even more expensive than the polymeric syringe.

Conventional syringes are used together with a hypodermic needle, which is connected to the syringe tip before use. These are cheaper to manufacture than the pre-filled syringes, but fail to completely empty the barrel/hypodermic needle of the medicament, i.e. a residual volume of the drug is lost for each injection. This leads to the loss of drugs comprising expensive active ingredients. Vials, carrying the medicament, are filled with a surplus of 20%, to compensate for loss of medicament in syringe dead space. Also, due to dead spaces in conventional syringes, air (in dead spaces before filling through needle) needs to be vented before injection, to prevent air embolism. This is a time consuming and medicament wasting procedure.

Moreover, multiple use of the same syringe to perform injections to more than one person causes the spread of several infectious diseases. It is estimated that over 2 million patients and users each year are infected with severe blood transmittable diseases, such as HIV, Hepatitis B and C due to the misuse of syringes. A majority of these injections are performed using conventional syringes or pre-filled conventional syringes available on the market.

Already in 2015, the World Health Organisation (WHO) set a time limit that all syringes worldwide should be exchanged for so called “smart syringes”, such as auto-disabled (AD) syringes, by 2020. Smart syringes prevent the reuse of the syringe, and thus decreases the spreading of the infectious diseases described above.

However, the smart syringes available on the market also suffer from the several drawbacks described above. For instance, the syringes fail to completely empty the barrel of the syringe of the medicament.

Hence, there is a need for an improved syringe which is inexpensive to manufacture and which decreases the residual volume of the medicament in the barrel after injection.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by proposing a syringe plunger rod extending along a longitudinal central axis comprising: a distal plunger top, having a sealing plug for sealing engagement with a syringe barrel; a proximal elongated shaft comprising a proximal end adapted to receive an applied force along an axis; wherein the sealing plug is made of an elastic material and wherein a sealing plug distal surface comprises concentric steps, wherein the concentric steps are climbing laterally.

A syringe barrel is also provided, said syringe barrel extending along a longitudinal central axis and comprising: a barrel cylinder defining a volume adapted to contain a drug, the barrel cylinder having a distal plunger socket wall and a tubular wall extending proximally from said distal plunger socket wall, a needle hub extending distally from the barrel cylinder, a hollow needle extending distally from the needle hub, wherein the needle hub is provided with longitudinal needle press-fit channel, wherein the press-fit channel is flush with the distal plunger socket wall, such that the press-fit channel extends proximally into the volume, wherein the hollow needle is press fitted in the press-fit channel.

A syringe comprising such syringe plunger rod and syringe barrel is also provided.

Further advantages will be apparent from the detailed description as well as the appended dependent claims.

### BRIEF DESCRIPTION OF THE DRAWINGS

These and other aspects, features and advantages of which the invention is capable, will be apparent and elucidated from the following description of embodiments of the present invention, reference being made to the accompanying drawings, in which

FIG. 1 is a longitudinal section of a syringe; and

FIG. 2 shows details of the syringe and sections of use positions of the syringe according to the present invention.

### DETAILED DESCRIPTION OF THE EMBODIMENTS

Embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

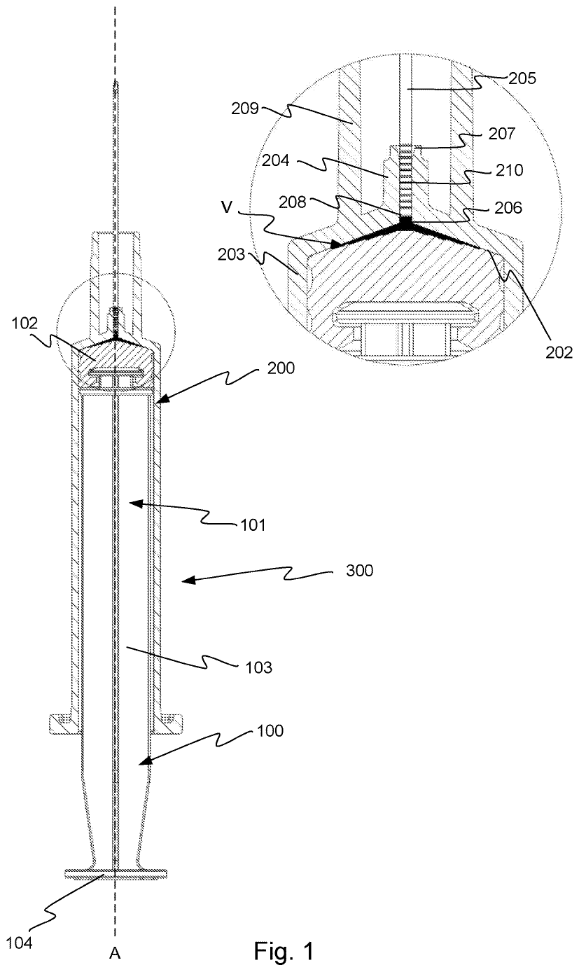

Referring to FIG. 1, a syringe plunger rod 100 is disclosed. The syringe plunger rod 100 extends along a longitudinal central axis A. The syringe plunger rod 100 comprises a distal plunger top 101. The distal plunger top 101 comprises a sealing plug 102. The sealing plug 102 seals against a syringe barrel 200, upon use, to form a syringe 300. Proximally of the sealing plug 102 an elongated shaft 103 is provided. At the proximal end of the elongated shaft 103 there is provided a push plate 104. The push plate 104 extends substantially transversally to the longitudinal axis A. The push plate 104 is adapted to receive an applied force along the axis A, upon use, such that the syringe plunger rod 100 is displaced in relation to the barrel 200.

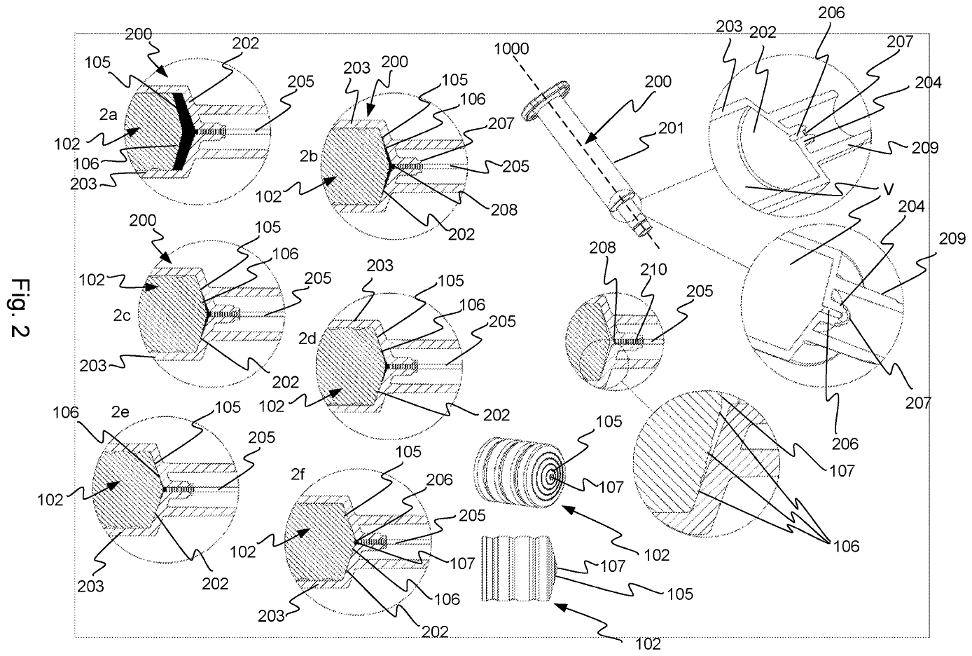

In accordance with FIG. 2, the present invention also provides a syringe barrel 200. The syringe barrel 200 extends along a longitudinal axis A. The syringe barrel 200 comprises a barrel cylinder 201 defining a volume V adapted to contain a drug. The barrel cylinder 201 has a distal plunger socket wall 202 and a tubular wall 203, extending proximally from said distal plunger socket wall 202. A needle hub 204 extends distally from the barrel cylinder 201. A hollow needle 205 extends distally from the needle hub 204. The needle hub 204 is provided with longitudinal needle press-fit channel 206. The press-fit channel 206 is flush with the distal plunger socket wall 202, such that the press-fit channel 206 extends proximally to the volume V, such that the press-fit channel 206 and falls out in volume V. The hollow needle 205 is press fitted in the press-fit channel 206. The wall thickness of the needle hub 204 around the press-fit channel 206 is preferably 0.5 to 1.5 mm, which provides stretch and flexibility properties during the needle mounting process. Further, the length of the press-fit channel 206 in the longitudinal direction is preferably 1.5 to 4.5 mm. The relatively thin thickness increases the seal between the hollow needle 205 and the needle hub 204, and decreases irregularities and weaknesses therein. Further, it minimises drag in the seal and provides a maximal contact diameter between the hollow needle 205 and the needle hub 204. Further, it provides a stretchability of the needle hub wall, which facilitates and aids the press-fitting of the hollow needle 205 inside the needle hub 204.

The sealing plug 102 is made of an elastic material. At the distal end of the sealing plug 102 a sealing plug distal surface 105 is arranged. The sealing plug distal surface 105 comprises concentric steps 106. In this context, the term “concentric” refers to an extension around the central axis A, and not necessarily with same step length, same step height, exact circles, exact same centre, at the circumference etc., while these are not excluded. In the embodiment disclosed in FIG. 2, this means that the different steps 106 are arranged at different radial distance from the central axis A and extends around the central axis A.

In the embodiment disclosed in FIG. 2, the number of concentric steps is five. However, this number may vary dependent on the size of the syringe in which the syringe plunger rod 100 is to be arranged and/or the medicament to be injected. The number of steps 106 may be from 2 to 50, such as for example two, three, four, five, six, seven, eight, nine, or ten in number.

The steps 106 are climbing laterally, i.e. towards the circumference of the sealing plug 102. When the syringe plunger rod 100 is displaced in relation to the barrel 200, eventually the sealing plug distal surface 105 will abut the distal plunger socket wall 202 of the barrel 200. Upon displacement of the syringe plunger rod 100 in relation to the barrel 200, the sealing plug 102 slides against the tubular wall 203 of the barrel 200. Since the steps 106 are climbing laterally, the most lateral step 106 will abut the distal plunger wall 202 first, squeezing the liquid to be injected centrally. In this way the last portion of the medicament to be injected, such as vaccine or viscous medicament is pushed centrally from the periphery into the needle channel. Due to the elasticity of the sealing plug 102, the sealing plug 102 will deform upon additional force in the distal direction, whereby the step centrally of the most lateral step 106 will be displaced in relation to the barrel 200 to abut the distal plunger socket wall 202. In this way the liquid to be injected is pushed further centrally. For each step 106 the same relative movement in relation to the barrel 200 is repeated to push the liquid centrally, and into the hollow needle 205 of the barrel 200. In this way, the amount of trapped air in the interface of the sealing plug 102 and the plunger socket wall 202 may be decreased.

The height of each step may be 0.01 mm to 1 mm. The number of steps 106 may vary from 2 to 50. The parameters regulating the step height and the number of steps may be the inner diameter of the barrel 200, the E module of the sealing plug 102. The width of the steps 106 may also vary.

Centrally, i.e. underneath the hollow needle 205, the sealing plug distal surface 105 is provided with a central plateau 107, from which the concentric steps climb laterally. The central plateau 107 is thus placed proximally directly underneath the hollow needle 205. In this way, the sealing plug distal surface 105 is configured such that a minimal amount of fluid to be injected is trapped in the barrel 200, since the plateau 107 may be arranged flush to the proximal end of the hollow needle 205.

The sealing plug distal surface 105 is generally conical, such that the most lateral concentric step 106 is arranged proximally a distal end point of the sealing plug 102, such as the plateau 107. This further helps the fluid to be injected to be pushed into the needle 205, since there will be a distal force component from the distally directed injection force.

Reverting to the syringe barrel 200, the needle hub 204 comprises a distal needle hub opening 207 which is tapered in the proximal direction, towards the longitudinal central axis A and a centre of the needle hub opening 207. The hollow needle 205 has a needle proximal end 208 with an external taper or rounding, tapered/rounded towards the longitudinal central axis A. A proximal needle end 208 of the hollow needle 205 is substantially flush with the distal plunger socket wall 202. The proximal end 208 of the hollow needle 205 is engaged in a needle hub 204. The internally taper of the needle hub 204 guides the hollow needle 205 into the needle hub 204 when displacing the hollow needle 205 into the press-fit channel 206 to attach the hollow needle 205 to the needle hub 204. In addition, the taper reduces the friction between the hollow needle 205 and the needle hub 204, facilitating the connection of the hollow needle 205. If the internal taper of the needle hub 204 and the external taper of the hollow needle 205 were straight and not tapered, it would be difficult to mate the needle proximal end 208 with the needle hub opening 207. Further, a high pressure would build up between the external surface of the needle proximal end 208 and the needle hub 204, such as the press-fit channel 206. A rounded external tapered on the needle proximal end 208 is advantageous since there is a decreased risk that the hollow needle 205 peels off material from the needle hub 204 during the mounting process. Sharp edges may scratch the needle hub 204, resulting in that plastic particles may be removed from the needle hub 204 and mix with the medicament to be injected through the hollow needle 205. The needle hub opening 207 and the remaining parts of the needle hub 204 are formed with one mould core, injection moulded or machined to form the needle hub 204 with its internal taper. Hence, there is no shoulder or other obstacle for the hollow needle 205 when inserted into the needle hub opening 207.

Due to the press-fitting of the hollow needle 205 into the needle hub 204, no adhesive or glue is required to attach the hollow needle 205 to the needle hub 204. In pre-filled syringes known in the art, both glass and/or polymer syringes, the use of an adhesive to connect the needle to the syringe is very common. During the injection moulding process when forming polymer syringes, the needle can be cast into the syringe barrel. However, this is not a cost efficient needle integration technique compared to the press fitting mounting process used for the syringe described herein. Moreover, all types of adhesive (such as epoxy resins or glues) used in a syringe incur a quality risk. The viscous adhesive may reach the drug/vaccine flow area within the syringe and thus damage the syringe. The press fitting omits the need for an adhesive. Also, there is always a risk that the hypodermic needle, once attached, may leak or detach during the injection process, e.g. if a Luer-slip syringe with a hypodermic needle is used. Finally, even when using smart syringes with hypodermic needles, it is possible to detach the hypodermic needle after an injection, and reuse the hypodermic needle together with another barrel, which contributes to spreading of infectious diseases. With the barrel 200 described herein, this issue is mitigated since the needle 205 is safely attached to the needle hub 204.

After press-fitting of the hollow needle 205 in the press-fit channel 206 it is preferred that the needle proximal end 208 is in proximo-distal level with the plunger socket wall 202, so as to minimize dead space in the syringe 300. The hollow needle 205 may have an inner diameter of about 0.15 to 0.2 mm.

The syringe barrel 200 is made of a polymeric material, preferably a plastic selected from Cyclo Olefin Polymer (COP), Cyclo Olefin Copolymer (COC), or polypropylene (PP). These polymers are specifically preferred, since they facilitate the press-fitting of the hollow needle 205 in the needle hub 204, since they have beneficial tensile strength characteristics.

A needle cover (not shown) may be arranged to a cover flange 209, said cover flange extending distally and being arranged laterally of the needle hub 204. The cover flange 209 is configured as a cylinder around the needle hub 204, and being configured such that the needle cover may be slipped into engagement onto the cover flange 209. The cover protects the user from the hollow needle 205. A cover flange 209 separated from the needle hub 204 allows for the cover to be dimensioned separately from the size of the needle hub 204, and vice versa. Also, the cover flange 209 may be made thinner in dimension which allows for an increased flexibility to facilitate the arrangement of the cover on the cover flange 209.

The hollow needle 205 has a plurality of circumferential recesses 210, such as radial grooves, on its outer surface, facing an internal cavity wall of the needle hub 204, once the hollow needle 205 is in position inside the needle hub 204. The circumferential recesses 210 increases the strength of the connection between the hollow needle 205 and the needle hub 204. This technique is simple and cost efficient to achieve a secure attachment of the hollow needle 205 inside the needle hub 204. The circumferential recesses 210 each has a V- or U-shaped cross-section, and the edges of the recesses 210 will engage the polymeric material of the needle hub 204 once the hollow needle 205 is press fitted in the needle hub 204. This is due to the fact that all polymeric materials have limited creep properties during press fitting. When subjected to press fitting, pressure, strain and tension is exerted on the solid polymeric needle hub 204, which starts to cold flow, and the circumferential recesses 210 having the edges can engage the needle hub 204. Hence, an increased adhesion of the hollow needle 205 in the needle hub 204 is achieved.

The needle hub opening 207 may be melted after the insertion of the hollow needle 205, such that is abuts the outer surface of the hollow needle 205. This technique allows for a lowered press fitting, and may also be used together with a needle without any circumferential recesses 210. The melted needle hub opening 207 together with the circumferential recesses 210 provides a stronger and more secure attachment of the needle 205 in the needle hub 205.

The barrel 200 has a volume V configured to contain a drug, a vaccine or any other type of pharmaceutical liquid or solution to be administered to a patient. The drug may be in liquid form to be injected into a patient or user.

The plunger rod 100 in FIG. 1 is disposed within the barrel 200. The plunger rod 100 extends along the tubular wall 203 and the axis A.

The sealing plug 102 is adapted to be in fluid-tight engagement with the tubular wall 203 of the barrel 200. By being in fluid-tight engagement with the tubular wall 203 of the barrel 200, the drug or liquid contained within the barrel 200 is prevented from leaking out of the syringe 300 in any other direction than through the hollow needle 205.

In FIGS. 2a to 2f, the operation of the syringe 300, comprising the plunger rod 100 and the barrel 200 of FIG. 1 is shown. FIGS. 2a to 2f show sections of use positions of the syringe 100.

In FIG. 2a the plunger rod 100 is pushed distally. The sealing plug 102 is displaced along the tubular wall 203 of the barrel 200. The sealing plug distal surface 105 has not yet abutted the plunger socket wall 202.

In FIG. 2b the periphery of the sealing plug distal surface 105, i.e. the most laterally placed step 106, abuts the most lateral part of the plunger socket wall. Hence, the fluid/medicament contained in volume V of the syringe 100 is forced centrally of the most lateral step 106.

In FIG. 2c the plunger rod 100 is advanced further distally in comparison with the position in FIG. 2b, and due to the elastic characteristics of the sealing plug 102, the sealing plug 102 is deformed to adapt to the shape of the plunger socket wall 202. Hence, the fluid/medicament contained in volume V of the syringe 100 is forced centrally of the step 106 being the second from the periphery.

In FIG. 2d the plunger rod 100 is advanced further distally in comparison with the position in FIG. 2c, and due to the elastic characteristics of the sealing plug 102, the sealing plug 102 is further deformed to adapt to the shape of the plunger socket wall 202. Hence, the fluid/medicament contained in volume V of the syringe 100 is forced centrally of the step 106 being the third from the periphery.

In FIG. 2e the plunger rod 100 is again advanced further distally in comparison with the position in FIG. 2d, and due to the elastic characteristics of the sealing plug 102, the sealing plug 102 is further deformed to adapt to the shape of the plunger socket wall 202. Hence, the fluid/medicament contained in volume V of the syringe 100 is forced centrally of the step 106 being the fourth from the periphery.

In FIG. 2f the plunger rod 100 is advanced yet further distally in comparison with the position in FIG. 2d, and due to the elastic characteristics of the sealing plug 102, the sealing plug 102 is further deformed to adapt to the shape of the plunger socket wall 202. Hence, the fluid/medicament contained in volume V of the syringe 100 is forced centrally of the step 106 being the fifth from the periphery. In this position the central plateau 107 closes the press-fit channel 206, such that the entire volume V of the barrel 200 has been emptied.

The sealing plug 102 is made from a resilient, flexible material, preferably a polymeric material, selected from a thermoplastic elastomer, or a natural or synthetic rubber material. The more force that is applied in the distal direction, the larger portion of the sealing plug distal surface 105 comes into contact with the plunger socket wall 202.

Further, the invention has mainly been described with reference to a few embodiments. However, as is readily understood by a person skilled in the art, other embodiments than the ones disclosed above are equally possible within the scope of the invention, as defined by the appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A syringe plunger rod extending along a longitudinal central axis, the syringe plunger rod comprising:
a distal sealing plug for sealing engagement with a syringe barrel;
a proximal elongated shaft comprising a proximal end adapted to receive an applied force along the longitudinal axis;
wherein the sealing plug is made of an elastic material;
wherein a sealing plug distal surface includes concentric steps; and
wherein the concentric steps climb laterally.

2. The syringe plunger rod according to claim 1, wherein the number of concentric steps is two, three, four, five, six, seven, eight, nine, or ten.

3. The syringe plunger rod according to claim 1, wherein the sealing plug distal surface is provided with a central plateau, from which the concentric steps climb laterally.

4. The syringe plunger rod according to claim 1, wherein the sealing plug distal surface is generally conical, such that a most lateral concentric step is arranged proximally a distal end point of the sealing plug.

5. A syringe barrel extending along a longitudinal central axis, the syringe barrel comprising:
a barrel cylinder defining a volume adapted to contain a drug, the barrel cylinder having a distal plunger socket wall and a tubular wall extending proximally from the distal plunger socket wall;
a needle hub extending distally from the barrel cylinder;
a hollow needle extending distally from the needle hub;
wherein the needle hub is provided with a longitudinal needle press-fit channel;
wherein the press-fit channel is flush with the distal plunger socket wall, such that the press-fit channel extends proximally into the volume;
wherein the hollow needle is press fitted in the press-fit channel.

6. The syringe barrel according to claim 5, wherein the needle hub comprises a needle hub opening having an internally tapered radius, tapered towards the longitudinal central axis and a center of the needle hub opening.

7. The syringe barrel according to claim 5, wherein the hollow needle has a needle proximal end with an externally tapered radius, tapered towards the longitudinal central axis and a center of a vertical cross-section of the hollow needle.

8. The syringe barrel according to claim 5, wherein the syringe barrel is fabricated from a polymeric material.

9. The syringe barrel according to claim 5, wherein a proximal needle end of the hollow needle is substantially flush with the distal plunger socket wall.

10. A syringe comprising:
a syringe plunger according to claim 1; and
a syringe barrel that includes
a barrel cylinder defining a volume adapted to contain a drug, the barrel cylinder having a distal plunger socket wall and a tubular wall extending proximally from the distal plunger socket wall,
a needle hub extending distally from the barrel cylinder, and
a hollow needle extending distally from the needle hub;
wherein the needle hub is provided with a longitudinal needle press-fit channel;
wherein the press-fit channel is flush with the distal plunger socket wall, such that the press-fit channel extends proximally into the volume;
wherein the hollow needle is press fitted in the press-fit channel.
